(19) United States
(12) Patent Application Publication
(10) Pub. No.: US 2024/0087718 A1
(43) Pub. Date: Mar. 14, 2024

(54) Method for Operating a System that Identifies a User's Eating Habits, Recommends Food, and Provides a Food Report

(71) Applicant: STAYJUNGLE Co., Ltd, Seoul (KR)

(72) Inventor: Eun Jung KIM, Seoul (KR)

(21) Appl. No.: 18/240,474

(22) Filed: Aug. 31, 2023

(30) Foreign Application Priority Data
Sep. 1, 2022 (KR) 10-2022-0110664

(51) Int. Cl. G16H 20/60; G16H 10/20

(52) U.S. Cl. CPC G16H 20/60; G16H 10/20

(57) ABSTRACT

Disclosed is a system operating method of identifying a type of user eating habits to recommend a food and provide a food report, the method including: receiving a request from a user to start chatting with a food recommendation chatbot operated by an account registered to a mobile messenger and providing food report content; presenting questions for identifying the type of user eating habits to the user, and analyzing the type of user eating habits based on responses entered by the user; providing a link to a result of the analyzed type of user eating habits to the user; receiving a request for a food recommendation from the user; providing a list of recommended foods reflecting the analyzed type of user eating habits to the user; providing nutritional information about a food, which is selected by the user from the list of recommended foods, to the user; and making a food report on the selected food and providing a link to the food report to the user, upon a response entered by the user to finally decide the food.

### (a) FIELD OF THE INVENTION

The disclosure relates to a system operating method of identifying the type of user eating habits to recommend a food and provide a food report, and more particularly to a system operating method of identifying the type of user eating habits to recommend food and provide a food report, in which an account registered to a mobile messenger and providing the food report operates a food recommendation chatbot to present questions for identifying the type of user eating habits to a user and provide a list of recommended foods reflecting the type of user eating habits analyzed based on responses entered by the user.

### (b) DESCRIPTION OF THE RELATED ART

Recently, modern people have been introduced to information related to foods through programs of various communication media, social networking services, etc., and have selected meal menus based on the introduced information. However, the introduced food information is often useless due to differences in likings, preferences and tastes for food between individuals.

Under the foregoing background, applications have been developed and used to improve a user's satisfaction by recommending suitable food to him/her. However, such applications recommend a user either food randomly selected without considering his/her preference or food simply highly preferred by many people. Besides, the food recommended by the applications falls into broad categories, and therefore such recommendation results may make it more difficult for a user to select food rather than providing high satisfaction to the user.

In this regard, Korean Patent Publication No. 2020-0118584 discloses “food recommendation system.”

### SUMMARY OF THE INVENTION

An aspect of the disclosure is to provide a system operating method of identifying the type of user eating habits to recommend food and provide a food report, in which an account registered to a mobile messenger and providing the food report operates a food recommendation chatbot to present questions for identifying the type of user eating habits to a user and provide a list of recommended foods reflecting the type of user eating habits analyzed based on responses entered by the user.

Further, another aspect of the disclosure is to provide a system operating method of identifying the type of user eating habits to recommend food and provide a food report, in which, when a user enters a response to finally decide a food selected from a given list of recommended foods, a food report on the selected food is made and a link to the food report is provided to the user.

In accordance with an embodiment of the disclosure, there is provided a system operating method of identifying a type of user eating habits to recommend a food and provide a food report, the method including: receiving a request from a user to start chatting with a food recommendation chatbot operated by an account registered to a mobile messenger and providing food report content; presenting questions for identifying the type of user eating habits to the user, and analyzing the type of user eating habits based on responses entered by the user; providing a link to a result of the analyzed type of user eating habits to the user; receiving a request for a food recommendation from the user; providing a list of recommended foods reflecting the analyzed type of user eating habits to the user; providing nutritional information about a food, which is selected by the user from the list of recommended foods, to the user; and making a food report on the selected food and providing a link to the food report to the user, upon a response entered by the user to finally decide the food.

Further, the presenting questions for identifying the type of user eating habits to the user, and the analyzing the type of user eating habits based on responses entered by the user through the food recommendation chatbot operated by the account providing the food report to the user may include presenting questions for identifying the type of user eating habits including at least one of preferred and non-preferred foods, non-preferred flavors, food allergies, food selection criteria, eating habit type, food selection types based on conditions, dietary goals, lifestyle, current health status, and personal physical information through the food recommendation chatbot.

Further, the presenting questions for identifying the type of user eating habits to the user, and the analyzing the type of user eating habits based on responses entered by the user through the food recommendation chatbot operated by the account providing the food report to the user may include calculating a type vector of user eating habits based on responses entered to the questions for identifying the type of user eating habits.

Further, the providing the list of recommended foods reflecting the analyzed type of user eating habits to the user may include calculating a similarity based on comparison between a previously stored type vector for each food and the calculated type vector of user eating habits, and providing the list of recommended foods, in which the foods are listed in order of calculated similarity.

Further, the providing nutritional information about a food, which is selected by the user from the list of recommended foods, to the user, may include providing the nutritional information about at least one of total content, calories, carbohydrates, protein, fat, cholesterol, and sodium for the food selected by the user.

Further, the making a food report on the selected food and the providing a link to the food report to the user, upon a response entered by the user to finally decide the food, may include providing at least one among a link to a site that sells the food finally selected by the user, information about a nearby store that sells the food selected by the user and reflects the current location of the user, and a link to a web page that contains information about a recipe for the food selected by the user.

Further, the making a food report on the selected food and the providing a link to the food report to the user, upon a response entered by the user to finally decide the food, may include making the food report to contain at least one among information about unit weight conversion for each ingredient used in the food finally selected by the user, information about the food, information about a relationship between the food and the ingredients, information about a relationship between the food and nutrients, and information about a relationship between the nutrients and efficacies.

Further, in the making a food report on the selected food and the providing a link to the food report to the user, upon a response entered by the user to finally decide the food, the food report may be made to contain the user's lifelog information and exercise information collected in association with a wearable device of the user.

### BRIEF DESCRIPTION OF THE DRAWINGS

FIG. 1 is a flowchart for describing the sequence of a system operating method of identifying the type of user eating habits to recommend a food and provide a food report.

FIG. 2 is a view for describing a process of analyzing the type of user eating habits in the system operating method of identifying the type of user eating habits to recommend a food and provide a food report.

FIG. 3 shows an exemplary image of analyzing the type of user eating habits in FIG. 2.

FIG. 4 is a view for describing a process of recommending a food to a user in the system operating method of identifying the type of user eating habits to recommend a food and provide a food report.

FIG. 5 shows an exemplary image of recommending a food to a user in FIG. 4.

FIG. 6 shows an exemplary image of a food report made in the system operating method of identifying the type of user eating habits to recommend a food and provide a food report.

FIG. 7 is a view for describing a structure of information included in a food report made in the system operating method of identifying the type of user eating habits to recommend a food and provide a food report.

### DETAILED DESCRIPTION

The disclosure may be variously changed and may have various embodiments, and thus specific embodiments will be illustrated in the accompanying drawings and described in detail.

However, it should be understood that those embodiments are not intended to limit the disclosure to the specific embodiments but include all changes, equivalents or alternatives within the spirit and scope of the disclosure. Like numerals refer to like elements throughout the accompanying drawings.

It should be understood that when an element is referred to as being “connected to” or “coupled to” another element, it may be directly connected to or coupled to another element or intervening elements may be present. On the other hand, when an element is referred to as being “directly connected to” or “directly coupled to” another element, there are no intervening elements present.

The terms used in the disclosure are merely used to describe specific embodiments and are not intended to limit the disclosure. A singular form includes a plural form as well, unless the context clearly dictates otherwise. In the disclosure, the term ‘include’ or ‘have’ may represent the presence of a feature, a number, a step, an operation, an element, a part or the combination thereof described in the disclosure, and may not exclude the presence or addition of another feature, another number, another step, another operation, another element, another part or the combination thereof.

Below, embodiments of the disclosure will be described in more detail with reference to the accompanying drawings. Hereinafter, the same reference numerals will be used for the same elements in the accompanying drawings, and repetitive descriptions of the same elements will be avoided.

First, according to the disclosure, a system for identifying the type of user eating habits to recommend a food and provide a food report may include a user, a mobile messenger, an application program interface (API) server, and a web browser.

A user refers to a person who activates a mobile messenger in a user terminal and chats with a food recommendation chatbot operated by an account through the account registered to the mobile messenger and providing food report content.

The mobile messenger refers to software that allows a user to send and receive a text, a file, a voice message through a wireless Internet network on a smartphone. Thus, the mobile messenger is downloaded and installed on the user terminal by a user and used in chatting with others.

The API server refers to a server that is built to recommend a food and provide a food report by identifying the type of user eating habits, in which a food-bio relationship network database (DB) may be established in the form of a graph DB or relational DB with data of linking cooking, recipes, ingredients, nutrition and efficacies and metadata such as papers supporting the data, and various implementation strategies such as a memory cache may be used to improve response performance.

The web browser refers to a program that allows a user to view an Internet web page of a food report made based on a food finally selected by the user and a result of analyzing the type of user eating habits provided according to the disclosure.

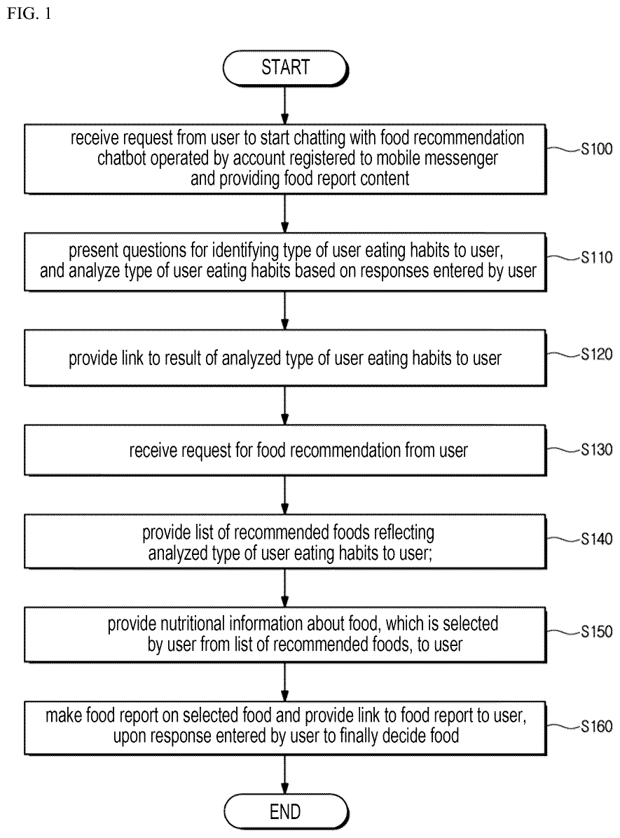

FIG. 1 is a flowchart for describing the sequence of a system operating method of identifying the type of user eating habits to recommend a food and provide a food report.

Referring to FIG. 1, in a system operating method of identifying the type of user eating habits to recommend a food and provide a food report according to the disclosure, a request is first received from a user to start chatting with a food recommendation chatbot operated by an account registered to a mobile messenger and providing food report content (S100).

Next, questions for identifying the type of user eating habits are presented to the user, and the type of user eating habits is analyzed based on responses entered by the user (S110).

In step S110, the food recommendation chatbot presents questions for identifying the type of user eating habits including at least one of preferred and non-preferred foods, non-preferred flavors, food allergies, food selection criteria, eating habits type, food selection types based on conditions, dietary goals, lifestyle, current health status, and personal physical information.

Further, in step S110, a type vector of user eating habits is calculated based on responses entered to the questions for identifying the type of user eating habits.

Next, a link to a result of the analyzed type of user eating habits is provided to the user (S120).

Next, a request for a food recommendation is received from the user (S130).

Next, a list of recommended foods reflecting the analyzed type of user eating habits is provided to the user (S140).

In step S140, a similarity is calculated based on comparison between a previously stored type vector for each food and the calculated type vector of user eating habits, and the list of recommended foods, in which the foods are listed in order of calculated similarity, is provided.

Next, nutritional information about the food selected by the user from the list of recommended foods is provided to the user (S150).

In step S150, there is provided the nutritional information about at least one of total content, calories, carbohydrates, protein, fat, cholesterol, and sodium for the food selected by the user.

Next, when the user enters a response to finally decide a food, a food report on the selected food is made and a link to the food report is provided to the user (S160).

In step S160, there is provided at least one among a link to a site that sells the food finally selected by the user, information about a nearby store that sells the food selected by the user and reflects the current location of the user, and a link to a web page that contains information about a recipe for the food selected by the user.

Further, in step S160, the food report is made to contain at least one among information about unit weight conversion for each ingredient used in the food finally selected by the user, information about the food, information about a relationship between the food and the ingredients, information about a relationship between the food and nutrients, and information about a relationship between the nutrients and efficacies.

In addition, in step S160, the food report may be made to contain the user's lifelog information and exercise information collected in association with a wearable device of the user.

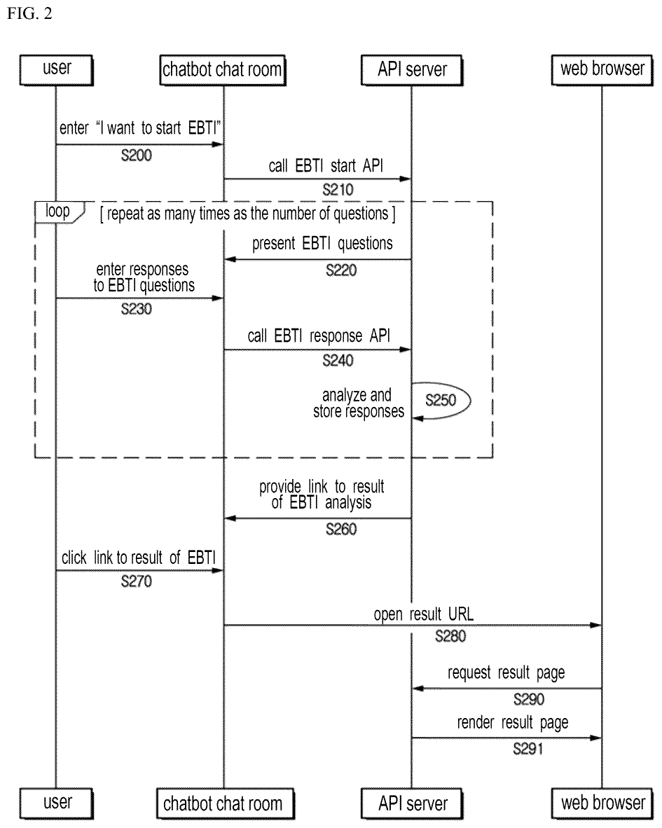

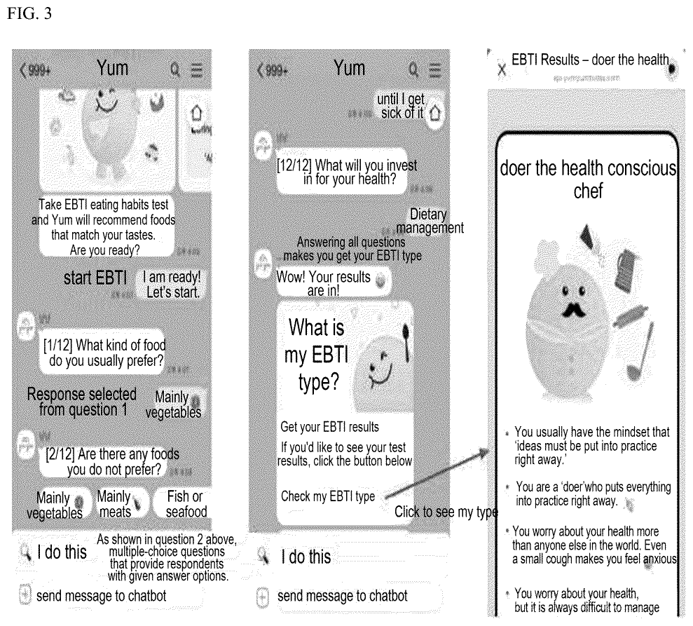

FIG. 2 is a view for describing a process of analyzing the type of user eating habits in the system operating method of identifying the type of user eating habits to recommend a food and provide a food report, and FIG. 3 shows an exemplary image of analyzing the type of user eating habits in FIG. 2.

Referring to FIGS. 2 and 3, in the process of analyzing the type of user eating habits according to the disclosure, a user first makes a request for analyzing the type of user eating habits (S200). In step S200, a user enters a request phrase such as ‘analyze my type of eating habits’ through a chat window.

Next, the food recommendation chatbot calls an API server to analyze the type of user eating habits (S210). In this case, step S210 may be repeated as many times as the number of questions for identifying the type of user eating habits.

Next, the food recommendation chatbot presents the questions for identifying the type of user eating habits as shown in FIG. 3 (S220).

In step S220, the questions for identifying the type of user eating habits are as follows. However, without limitations, the following questions, and may be continuously supplemented, and new questions may be added thereto.

[Examples of Questions]
Question 1. What kind of food do you usually prefer?
1) Mainly vegetables 2) Mainly meats 3) Fish or seafood 4) All of the above
Question 2. Are there any foods you do not prefer?
1) Mainly vegetables 2) Mainly meats 3) Fish or seafood 4) None of the above
Question 3. What kind of food do you eat when you are under stress?
1) Spicy food 2) Fatty food 3) Sour food 4) Sweet food
Question 4. What flavor do you not prefer?
1) Fishy taste 2) Sweet taste 3) None of the above
Question 5. What are your criteria for choosing food?
1) Tasty 2) Nutritionally healthy
Question 6. What would you do when you were full but had food left?
1) I stop eating because I am full 2) I eat it all because it is a waste.
Question 7. We are living in the busy modern society. What do you think as lunch time approaches?
1) I can't wait! What to eat today? 2) Annoying! Can't I take a pill as a lunch substitute?
Question 8. What do you do when there is nothing to eat at home?
1) Cooking anything quickly 2) Searching for the delivery app right away 3) Not eating anything because it's a hassle
Question 9. What are you doing for your health this month?
1) Exercise 2) Meal delivery 3) Nutritional supplement once a day 4) None of the above
Question 10. What is my meal these days?
1) Delivery food 2) Home-cooked meal
Question 11. The trendy 00 is so delicious. If you are obsessed with one food, what are you like?
1) I tend to eat until I get sick of it. 2) Trying it once is enough.
Question 12. What will you invest in for your health?
1) Dietary management 2) Nutrients

Next, the user enters responses to the questions for identifying the type of user eating habits (S230).

Next, the food recommendation chatbot calls the API server to analyze the responses entered by the user to the questions for identifying the type of user eating habits (S240).

Next, the API server analyzes and stores the type of user eating habits based on the response entered by a user (S250).

In step S250, the type vector of user eating habits is calculated based on the responses entered to the questions for identifying the type of user eating habits.

Next, the food recommendation chatbot provides the user with a link to the results of the type of user eating habits analyzed by the API server (S260).

Next, the user clicks the link to the result of the analyzed type of user eating habits (S270).

Next, the food recommendation chatbot makes the web browser open the clicked link to the result of the analyzed type of user eating habits (S280, S290, S291).

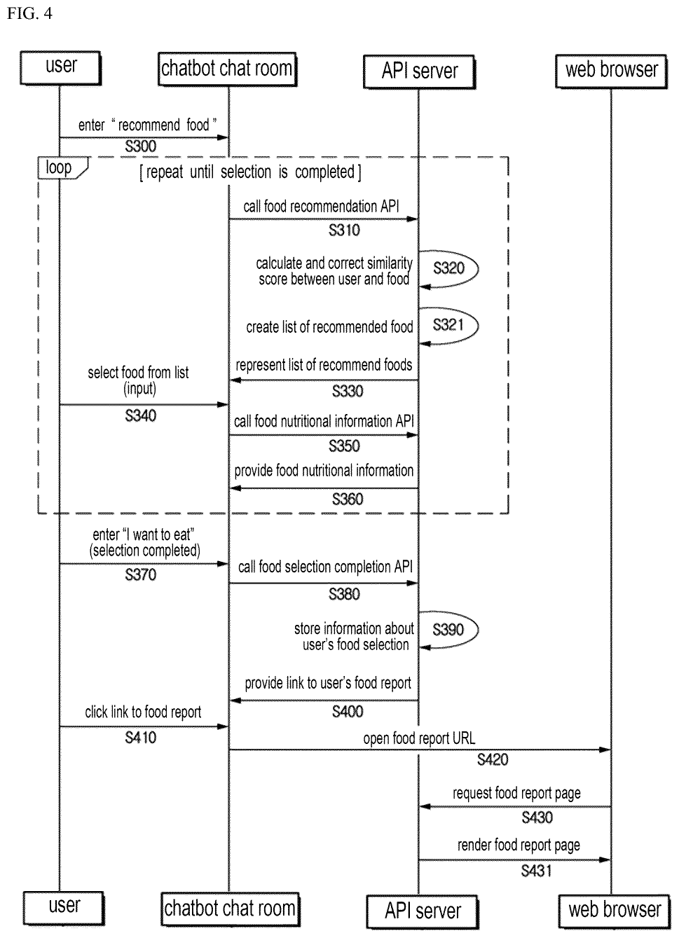

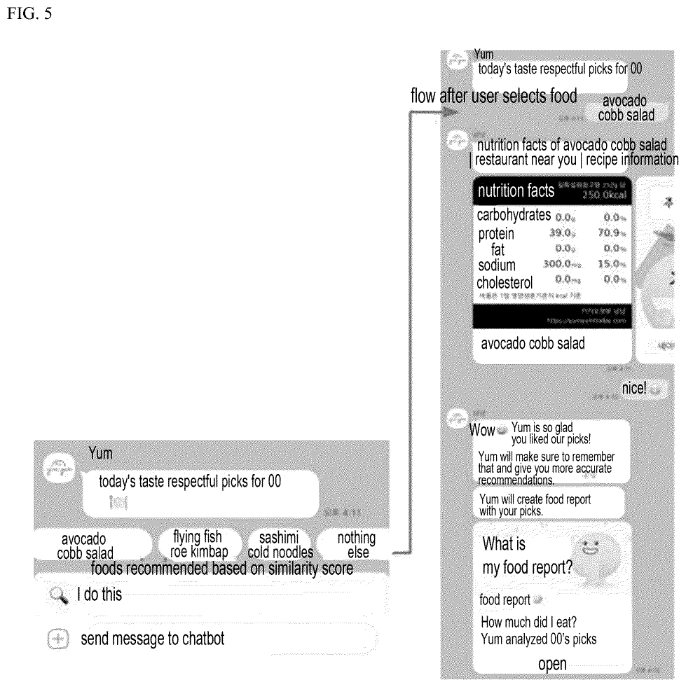

FIG. 4 is a view for describing a process of recommending a food to a user in the system operating method of identifying the type of user eating habits to recommend a food and provide a food report, and FIG. 5 shows an exemplary image of recommending a food to a user in FIG. 4.

Referring to FIGS. 4 and 5, in the process of recommending a food according to the disclosure, a food recommendation is first requested by a user (S300). In step S300, the user enters a request phrase such as “recommend food” through the chat window.

Next, the food recommendation chatbot calls the API server for the food recommendation (S310). In this case, step S310 may be repeated until the user finally selects a food.

Next, the API server calculates a similarity based on comparison between a previously stored type vector for each food and a calculated type vector of user eating habits, and creates a list of recommended foods, in which the foods are listed in order of calculated similarity (S320, S321).

Next, the food recommendation chatbot receives and provides the created list of recommended foods as shown in FIG. 5 (S330).

Next, the user selects one a food from the provided list of recommended foods and inputs the selected food to the food recommendation chatbot (S340).

Next, the food recommendation chatbot calls the API server to provide nutritional information about the selected food (S350).

Next, as shown in FIG. 5, the food recommendation chatbot receives the nutritional information, such as at least one among total content, calories, carbohydrates, protein, fat, cholesterol, and sodium for the food selected by the user, from the API server and provides the received nutritional information (S360).

Next, the user enters a response to finally decide a food (S370).

In step S370, the user enters a response phrase such as ‘I want to eat’ through the chat window.

Next, the food recommendation chatbot calls the API server to provide information about the finally selected food (S380).

Next, the API server stores the information about the food finally selected by the user and makes a food report based on that food (S390).

In step S390, the food report is made to contain at least one among information about unit weight conversion for each ingredient used in the food finally selected by the user, information about the food, information about a relationship between the food and the ingredients, information about a relationship between the food and nutrients, and information about a relationship between the nutrients and efficacies. In this case, in step S390, when there are the user's lifelog information and exercise information collected in association with a wearable device of the user, the food report may be made reflecting the user's lifelog information and exercise information.

Next, as shown in FIG. 5, the food recommendation chatbot provides the user with the link to the food report made by the API server (S400).

Next, the user clicks the link to the provided food report (S410).

Next, the food recommendation chatbot makes the web browser open the clicked link to the food report (S420, S430, S431).

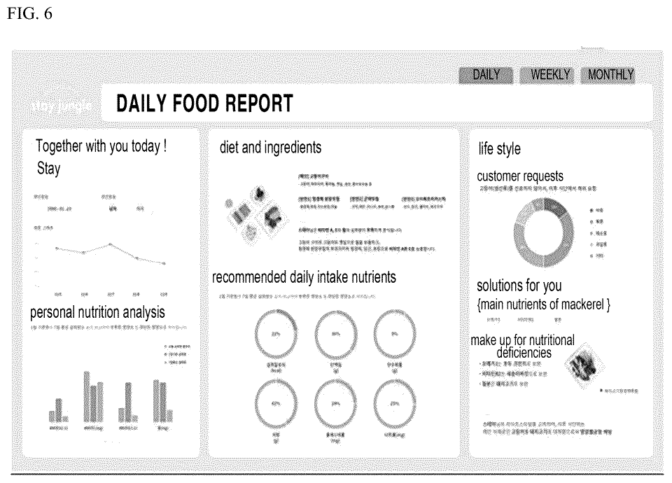

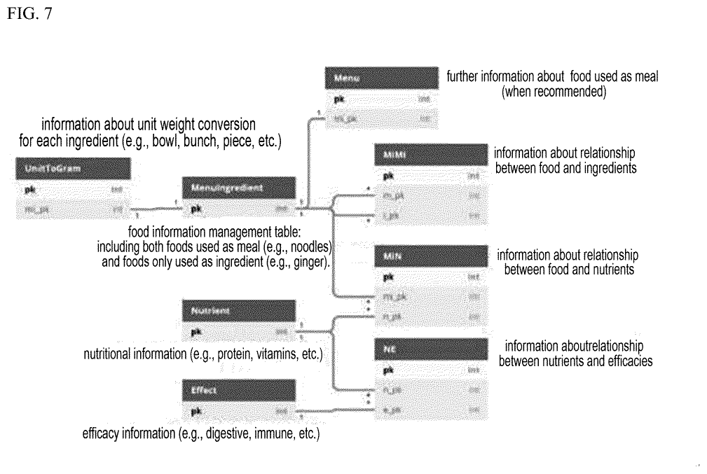

FIG. 6 shows an exemplary image of a food report made in the system operating method of identifying the type of user eating habits to recommend a food and provide a food report, and FIG. 7 is a view for describing a structure of information included in a food report made in the system operating method of identifying the type of user eating habits to recommend a food and provide a food report.

Referring to FIGS. 6 and 7, the food report according to the disclosure may provide information such as a personal nutrition analysis, diets and ingredients, recommended daily intake nutrients, a lifestyle, and dietary solutions for the user, which may be provided daily, weekly, or monthly depending on the user's settings. In other words, as shown in FIG. 7, the food report contains at least one among information about unit weight conversion for each ingredient used in the food finally selected by the user, information about the food, information about a relationship between the food and the ingredients, information about a relationship between the food and nutrients, and information about a relationship between the nutrients and efficacies.

More specifically, in the food report, a preference score is assigned to each food based on the user's health data and preference data (for example, the higher the score, 1) the more the effects that match the health data, 2) the more the preferred ingredients, and 3) the less the avoided ingredients).

Further, in the food report, an overall dietary pattern is generated with reference to preferred types of dishes (Korean food vs. Western food) based on lifestyle data and preference data (for example, when the user prefers the Western food, a dietary pattern of Korean-Western-Western-Korean-Western-Western is generated)

Further, in the food report, only main dishes are extracted from the finally selected foods and then arranged according to the generated pattern.

Further, in the food report, two side dishes, of which ingredients or main flavor components do not overlap with those of each main dish, may be selected and combined.

The functional operations described in this specification and the embodiments of the subject matters may be implemented in digital electronic circuits, computer software, firmware or hardware including the structures disclosed in this specification and their structural equivalents, or in one or more combinations thereof.

The embodiments of the subject matter described in this specification may be implemented as one or more computer program products, i.e., one or more modules for computer program instructions encoded on a tangible program medium to be executed by or to control the operations of a data processing device. The tangible program medium may be a radio signal or a computer readable medium. The radio signal refers to an artificially generated signal, such as a machine-generated electrical, optical or electromagnetic signal, which is generated to encode information to be transmitted to a suitable receiving device and executed by a computer. The computer readable medium may be a machine-readable storage device, a machine-readable storage substrate, a memory device, a combination of materials that affects a machine-readable radio signal, or one or more combinations thereof.

A computer program (also known as a program, software, a software application, a script, or a code) may be written in any form of a programming language including a priori or procedural language or a compiled or interpreted language, and may be developed in any form including a stand-alone program or module, a component, a subroutine, or other units suitable for use in a computer environment.

The computer program does not necessarily correspond to files of a file device. The program may be stored in a single file provided to a requested program, multiple interacting files (e.g., files that store parts of one or more modules, subordinate programs or codes), or parts of files containing other programs or data (e.g., one or more scripts stored in a markup language document).

The computer program may be developed to run on a single computer or multiple computers located at a single site or distributed across a plurality of sites and interconnected by a communication network.

In addition, the logic flows and structural block diagrams described in this specification, which describe the corresponding operations and/or specific methods supported by the corresponding functions and steps supported by the disclosed structural means, may also be used in build the corresponding software structure and algorithm and their equivalents.

The processes and logic flows described in this specification may be implemented by one or more programmable processors that execute one or more computer programs to perform the functions by operating on the received data and generating the outputs.

The processors suitable for executing the computer programs include, for example, any one or more processors of both general-purpose and special-purpose microprocessors and any type of digital computer. In general, the processor may receive instructions and data from a read-only memory, a random-access memory, or both of them.

The essential elements of the computer are one or more memory devices for storing instructions and data, and a processor for executing the instructions. In addition, the computer may generally couple with or include the devices to receive data from one or more devices for storing data, such as magnetic, magneto-optical, or optical disks, transmit the data to the devices, or perform both receiving and transmitting operations. The computer need not have such devices.

The foregoing description presents the best mode of the disclosure, and provides examples to describe the disclosure and to enable those skilled in the art to make and use the disclosure. In this specification, the disclosure is not limited to the specific terms.

Although the disclosure has been described in detail with reference to the foregoing examples, those skilled in the art may apply modifications, alterations and changes to these examples without departing from the scope of the disclosure. In short, it is not necessary to include all the functional blocks shown in the accompanying drawings separately, or to follow all the sequences shown in the accompanying drawings in the shown order so as to achieve the intended effect of the disclosure, and it is noted that other functional blocks or other sequences may fall within the technical scope of the disclosure described in the appended claims.

As described above, a system operating method of identifying the type of user eating habits to recommend a food and provide a food report according to the disclosure presents questions for identifying the type of user eating habits to the user through a food recommendation chatbot operated by an account registered to a mobile messenger and providing food report content, and provides a list of recommended foods reflecting the type of user eating habits analyzed based on responses entered by the user, thereby having effects on more correctly recommending a food to a user based on the type of user eating habits and the preferred food.

Further, according to the disclosure, when a response is entered by the user to finally decide the food, a food report on the selected food is made and a link to the food report is provided to the user, thereby creating a personal dietary pattern customized for the user's body based on information about unit weight conversion for each ingredient used in the food finally selected by the user, information about the food, information about a relationship between the food and the ingredients, information about a relationship between the food and nutrients, and information about a relationship between the nutrients and efficacies.

What is claimed is:

1. A system operating method of identifying a type of user eating habits to recommend a food and provide a food report, the method comprising:
receiving a request from a user to start chatting with a food recommendation chatbot operated by an account registered to a mobile messenger and providing food report content;
presenting questions for identifying the type of user eating habits to the user, and analyzing the type of user eating habits based on responses entered by the user;
providing a link to a result of the analyzed type of user eating habits to the user;
receiving a request for a food recommendation from the user;
providing a list of recommended foods reflecting the analyzed type of user eating habits to the user;
providing nutritional information about a food, which is selected by the user from the list of recommended foods, to the user; and
making a food report on the selected food and providing a link to the food report to the user, upon a response entered by the user to finally decide the food.

2. The method of claim 1, wherein the presenting questions for identifying the type of user eating habits to the user, and the analyzing the type of user eating habits based on responses entered by the user through the food recommendation chatbot operated by the account providing the food report to the user comprise
presenting questions for identifying the type of user eating habits including at least one of preferred and non-preferred foods, non-preferred flavors, food allergies, food selection criteria, eating habits type, food selection types based on conditions, dietary goals, lifestyle, current health status, and personal physical information through the food recommendation chatbot.

3. The method of claim 1, wherein the presenting questions for identifying the type of user eating habits to the user, and the analyzing the type of user eating habits based on responses entered by the user through the food recommendation chatbot operated by the account providing the food report to the user comprise
calculating a type vector of user eating habits based on responses entered to the questions for identifying the type of user eating habits.

4. The method of claim 1, wherein the providing the list of recommended foods reflecting the analyzed type of user eating habits to the user comprises
calculating a similarity based on comparison between a previously stored type vector for each food and the calculated type vector of user eating habits, and providing the list of recommended foods, in which the foods are listed in order of calculated similarity.

5. The method of claim 1, wherein the providing nutritional information about a food, which is selected by the user from the list of recommended foods, to the user, comprises
providing the nutritional information about at least one of total content, calories, carbohydrates, protein, fat, cholesterol, and sodium for the food selected by the user.

6. The method of claim 1, wherein the making a food report on the selected food and the providing a link to the food report to the user, upon a response entered by the user to finally decide the food, comprise
providing at least one among a link to a site that sells the food finally selected by the user, information about a nearby store that sells the food selected by the user and reflects the current location of the user, and a link to a web page that contains information about a recipe for the food selected by the user.

7. The method of claim 1, wherein the making a food report on the selected food and the providing a link to the food report to the user, upon a response entered by the user to finally decide the food, comprise
making the food report to contain at least one among information about unit weight conversion for each ingredient used in the food finally selected by the user, information about the food, information about a relationship between the food and the ingredients, information about a relationship between the food and nutrients, and information about a relationship between the nutrients and efficacies.

8. The method of claim 1, wherein in the making a food report on the selected food and the providing a link to the food report to the user, upon a response entered by the user to finally decide the food,
the food report is made to contain the user's lifelog information and exercise information collected in association with a wearable device of the user.
